(19) United States
(12) Patent Application Publication
(10) Pub. No.: US 2010/0211050 A1
(43) Pub. Date: Aug. 19, 2010

(54) Coupling arrangement for a telescopic device

(75) Inventor: Preben Luther, Kokkedal (DK)

Correspondence Address: COLOPLAST A/S;Attention: Corporate Patents, Holtedam 1, DK-3050 Humlebaek, DK

(73) Assignee: COLOPLAST A/S, Humlebaek (DK)

(21) Appl. No.: 12/600,274

(22) PCT Filed: May 16, 2008
(86) PCT No.: PCT/DK08/50110

§ 371 Date: May 3, 2010

(30) Foreign Application Priority Data
May 16, 2007 (DK) PA 2007 00737
May 16, 2007 (US) US60924466

(51) Int. Cl. A61M 27/00

(52) U.S. Cl. 604/544

(57) ABSTRACT

A telescopic device (1) comprising a first tubular element and an extension member displaceably arranged in an axial direction within a first passageway (5) of the first tubular element, wherein the extension member is provided with a first radial protruding part (8) on a first section of the extension member and that the first section is adapted to be changed between; a first radial extent (8) of the first radial protruding part having a larger dimension than the radial extent (13) of at least a part of the first passageway and; a second radial extent (9) of the first radial protruding part having a radial extent equal to or smaller than the radial extent of at least a part of the first passageway.

### TECHNOLOGY FIELD

The present invention relates to a telescopic device and a coupling arrangement for coupling the telescopic device in an extended configuration. In particular the invention relates to a telescopic catheter and a coupling arrangement for coupling the telescopic device in a ready-to-use configuration.

### BACKGROUND

The use of intermittent catheters has become almost a standard for persons not able to urinate of free will. Such users, typically paralysed persons such as para- and tetraplectics, have found that using intermittent catheters has greatly improved their freedom to move around and lead an active life active as catheterisation can be performed anywhere.

However, in order for the user to come out publicly and socialize it has become more and more important that such products are discreet and easy to carry around. Thus, a demand for compact catheters, which can easily be stored and carried around in handbags or pockets devices has grown.

In order to fulfil such needs products such as the SpeediCath® Compact produced by Coloplast A/S has been developed. However, this product mainly targets female users. Male users have a much longer urinary channel and thus other demands and requirements are to be fulfilled for a male product.

Many of these issues and solutions thereto have been discussed in WO 2006/045809, which discloses an expandable catheter with a transition between the individual sections allowing insertion of the transition into urethra.

However, there is still a need for alternative and improved solutions as will be discussed herein.

### SUMMARY OF THE INVENTION

The present invention relates to a telescopic device comprising a first tubular element and an extension member displaceably arranged in an axial direction within a first passageway of the first tubular element, wherein the extension member is provided with a first radial protruding part on a first section of the extension member and that the first section is adapted to be changed between; a first radial extent of the first radial protruding part having a larger dimension than the radial extent of at least a part of the first passageway and; a second radial extent of the first radial protruding part having a radial extent equal to or smaller than the radial extent of at least a part of the first passageway.

This provides a telescopic device with simple engagement means in the form of the radial protruding part, which will couple with the extension member and the first tubular element. As the first radial protruding part has a first cross sectional dimension which is larger than the cross sectional dimension of the first passageway it is prevented that the extension member can be pushed back into the first tubular element.

The term ‘radial extent’ should be understood as the dimension from the axis of the part to outer most point (relative to the axis) on that part in any radial direction from said axis.

In one embodiment of the present invention the first radial extent of the first radial protruding part may be engaged with an external portion of the first tubular element when the telescopic device is in its extended position. This means that when the telescopic device is brought from its collapsed position into its extended position, the first radial protruding part is manoeuvred from inside the first tubular element to the outside of the first tubular element, and when the extension member is manoeuvred towards the telescopic device's collapsed position, the first radial extent of the firs radial protruding part engages the proximal end of the first tubular element on the external surface of the proximal end.

In one embodiment of the present invention the first radial extent of the first radial protruding part may be engaged with an inner portion of the first tubular element when the telescopic device is in its collapsed position. This means that when the telescopic device is in its collapsed position the first radial protruding part is inside the first tubular element. When the telescopic device is in the transitional phase between being in a complete collapsed position and a complete extended position, the first radial protruding part is inside the first tubular element. When the telescopic device is being manoeuvred from its collapsed position towards its extended position and as soon as no part of the first radial protruding part is inside the first tubular element, the telescopic device has been locked in its extended position.

The second radial extent of the first radial protruding part ensures that the extension member and the first radial protruding part cannot be manoeuvred completely outside the first tubular element. Therefore, the second radial extent may operate as a blocking device. During the normal operation of the telescopic device, the second radial extent does not exit the proximal end of the first tubular element.

This means that the when the telescopic device is in its extended position, the first radial extent is outside the first tubular element while the second radial extent is inside the first tubular element.

In one embodiment the first radial protruding part is an annular rib formed around the extension member.

Typically, the first radial protruding part, for example provided as above-mentioned rib, has a proximal surface and a distal surface, facing opposite directions along the axis of the telescopic device, wherein the proximal surface forms a smaller angle with the axis of the telescopic device than the distal surface.

This results in that the extension member is easier, i.e. uses less force, to pull in the direction of the proximal surface than in the direction of the distal surface.

In one embodiment the first radial protruding part is formed of a compressible material, for example polyurethane.

Thus, deformation of the first radial protruding part may be provided by compressing the material.

Other compressible materials may for example also be used, such as different thermoplastic materials, rubbers or foams.

In yet another embodiment the proximal end of the first tubular element has a decreased inner circumference, and that the extension member is provided with a first rim at the distal end of the extension member, where the outer circumference of the first rim is larger than the decreased inner circumference of the proximal end of the tubular element.

Thus, it will not be possible to pull the extension member all the way through the first tubular element as the first flange will lock against the decreased inner circumference.

The telescopic device may in one embodiment be formed so that the axial length of the decreased inner circumference is smaller than the axial distance between the first flange and the radial protruding part. Thus, the radial protruding part can be pulled all the way through and abut against the proximal end of the first tubular element.

In order to further secure that the extension member is not pushed back into the first tubular element after it has been used the first cross section can be formed with a dimension equal to or larger than the cross section of the first tubular element.

In one embodiment the telescopic device may be adapted to allowing fluid communication. Thus, the extension member can be a second tubular element.

This may for example be the case in embodiments where the a telescopic intermittent urinary catheter, wherein the first tubular element can be the distal section and the second tubular element can be the proximal section. This allows urine to flow through both telescopic sections, typically from the bladder through the proximal and out through the distal section.

In one embodiment of the telescopic intermittent urinary catheter at least a part of the first passageway of the proximal end of the distal section is formed as a narrowing part.

### BRIEF DESCRIPTION OF THE DRAWINGS

The invention will be discussed further with reference to the following example embodiments, wherein

FIG. 1 shows in section the transition area between the proximal section and the distal section of a telescopic catheter according to the invention where the coupling element is in a partially collapsed configuration, and

FIG. 2 shows in section the transition area between the proximal section and the distal section of a telescopic catheter according to the invention where the coupling element is in a extended configuration.

### DETAILED DESCRIPTION

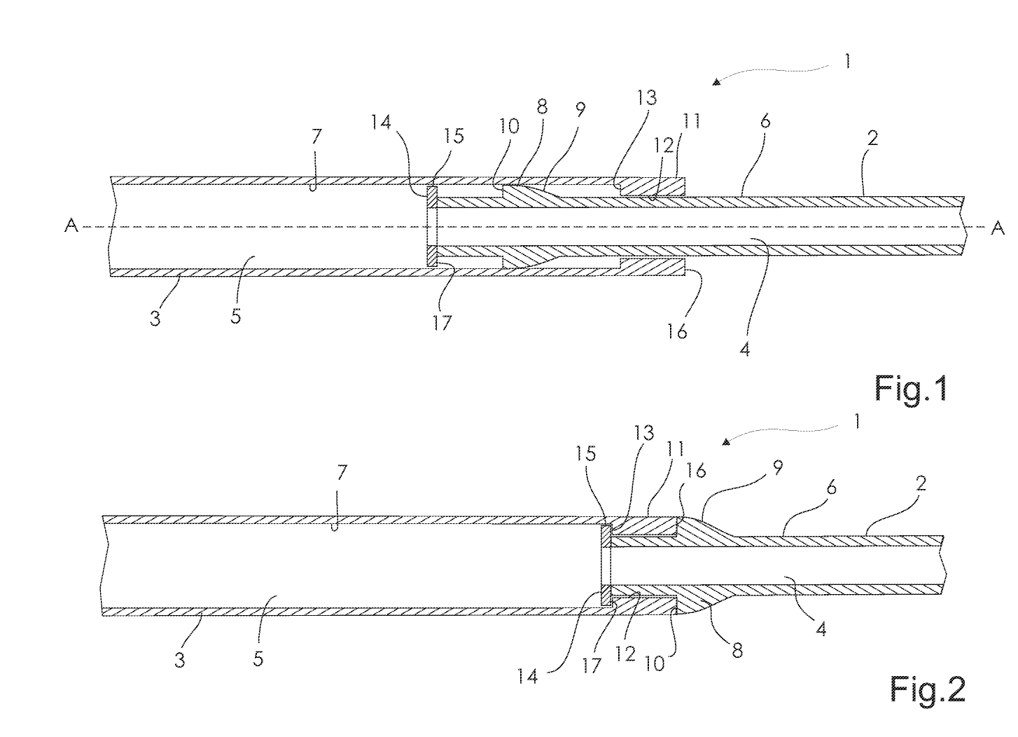

A telescopic intermittent catheter 1 is shown partly and in section in FIG. 1 around a first axis A-A. The catheter is formed of a proximal section 2 (corresponding to the extension member described above) and a distal section 3 (corresponding to the first tubular element described above).

Both sections are formed as tubular elements, wherein the distal section 3 defines a first passageway 5 and the proximal section 2 defines a second passageway 4 through which urine may flow in a flow direction from the first proximal section to the distal section during use.

The outer surface 6 of the proximal section 2 has a circumference, which is smaller than the circumference of the inner surface 7 of the distal section 3, so that the proximal section 2 at least partly can be displaceable placed within the first passageway 5.

On the proximal surface there is provided an annular rib 8, which in FIG. 1 is compressed to fit into the first passageway 5. The rib 8 is formed with a tapering surface 9 on its proximal side and a vertical surface 10 on its distal side. As can be seen the tapering surface 9 forms a smaller angle with the axis A-A than the vertical surface 10. This allows for a user to easily pull the distal section and the proximal section from a collapsed configuration as shows in FIG. 1 to an expanded configuration as shown in FIG. 2. Should the user attempt to push the part into a collapsed position the rib will expand as pressure is applied to the vertical surface and create a much tighter engagement that if the two section were pulled towards their expanded configuration.

At the proximal end 11 of the distal section 3 there is provided a narrowing part 12 having a smaller circumference than the circumference of the inner surface of the distal section 3. This change in circumference provides an edge 13 at the transition between the inner surface 7 and the narrowing part 12.

On the distal side of the annular rib 8 there is provided a rim 14. The rim has an outer surface 15 with a circumference, which is smaller than the circumference of the inner surface 7 and larger than the circumference of the narrowing part 12. The rim is formed of a material, which is stiffer than the material of the rib 8. Alternatively or additionally the rim may have a larger axial extent than the rib 8, which stabilises the rim making it less susceptible to deformation.

The radial extent between the rib and the rim is larger than the radial extent of the narrowing part 12. Thus, when the distal section and the proximal section are pulled into their expanded configuration the narrowing part 12 will engage between the rib and the rim locking the telescopic catheter in its expanded configuration.

As can be seen in FIG. 2 the vertical surface 10 faces the proximal end surface 16 of the distal section and the edge 13 faces the proximal surface 17 of the rim. These surfaces facing each other provide a tight engagement, which prevents the expanded telescopic catheter to be collapsed or separated unintentionally.

1. A telescopic device comprising a first tubular element and an extension member displaceably arranged in an axial direction within a first passageway of the first tubular element, wherein the extension member is provided with a first radial protruding part adapted to be changed between,
a first radial extent of the first radial protruding part having a larger dimension than the radial extent of at least a part of the first passageway and,
a second radial extent of the first radial protruding part having a radial extent equal to or smaller than the radial extent of at least a part of the first passageway.

2. A telescopic device according to claim 1, wherein the first radial extent of the first radial protruding part is engaged with an external portion of the first tubular element when the telescopic device is in its extended position.

3. A telescopic device according claim 1, wherein the first radial extent of the first radial protruding part is engaged with an inner portion of the first tubular element when the telescopic device is in its collapsed position.

4. A telescopic device according to claim 1, wherein the first radial protruding part is an annular rib formed around the extension member.

5. A telescopic device according to claim 1, wherein the first radial protruding part has a proximal surface and a distal surface, facing opposite directions along the axis of the telescopic device, wherein the proximal surface forms a smaller angle with the axis of the telescopic device than the distal surface.

6. A telescopic device according to claim 1, wherein the first radial protruding part is formed of a compressible material.

7. A telescopic device according to claim 6, wherein the compressible material is polyurethane.

8. A telescopic device according to claim 1, wherein a proximal end of the first tubular element has a decreased inner circumference, and the extension member is provided with a first rim at a distal end of the extension member, where an outer circumference of the first rim is larger than the decreased inner circumference of the proximal end of the tubular element.

9. A telescopic device according to claim 8; wherein a first cross section of the extension member has a dimension equal to or larger than a cross section of the first tubular element.

10. A telescopic device according to claim 9, wherein the extension member is a second tubular element.

11. A telescopic device according to claim 10, wherein the telescopic device is a telescopic intermittent urinary catheter.

12. A telescopic device according to claim 11, wherein the first tubular element is a distal section and the second tubular element is a proximal section of the telescopic intermittent urinary catheter.

13. A telescopic device according to claim 12, wherein at least a part of the first passageway of the proximal end of the distal section is formed as a narrowing part.
